(19) United States
(12) Patent Application Publication
(10) Pub. No.: US 2018/0242898 A1
(43) Pub. Date: Aug. 30, 2018

(54) VIEWING STATE DETECTION DEVICE, VIEWING STATE DETECTION SYSTEM AND VIEWING STATE DETECTION METHOD

(71) Applicant: PANASONIC INTELLECTUAL PROPERTY MANAGEMENT CO., LTD., Osaka (JP)

(72) Inventors: Masatoshi MATSUO, Fukuoka (JP); Tsuyoshi NAKAMURA, Fukuoka (JP); Tadanori TEZUKA, Fukuoka (JP)

(73) Assignee: PANASONIC INTELLECTUAL PROPERTY MANAGEMENT CO., LTD., Osaka (JP)

(21) Appl. No.: 15/747,651

(22) PCT Filed: Aug. 8, 2016
(86) PCT No.: PCT/JP2016/003640

§ 371 Date: Jan. 25, 2018

(30) Foreign Application Priority Data
Aug. 17, 2015 (JP) 2015-160546

(51) Int. Cl. A61B 5/16; A61B 5/00; G06K 9/00; G09B 5/02

(52) U.S. Cl. CPC A61B 5/165; A61B 5/0077; A61B 5/0013; G06K 9/00624; A61B 2576/00; G06K 9/00302; G09B 5/02; A61B 2503/12; G06K 9/00228

(57) ABSTRACT

A viewing state detection device (6) is configured to include an image input unit (11) to which temporally consecutive captured images including an audience and information on the captured time of the captured images are input, an area detector (12) that detects a skin area of the audience from the captured images, a vital information extractor (13) that extracts vital information of the audience based on time-series data of the skin area, a viewing state determination unit (17) that determines the viewing state of the audience based on the vital information, a content information input unit (14) to which content information including at least temporal information of the content is input, and a content viewing state storage unit (19) that stores the viewing state in association with the temporal information of the content.

### TECHNICAL FIELD

The present disclosure relates to a viewing state detection device, a viewing state detection system, and a viewing state detection method for detecting viewing states such as a degree of concentration and drowsiness of an audience viewing a content based on vital information of the audience detected in a non-contact manner using a camera.

### BACKGROUND ART

In recent years, a technique for estimating the psychological state of a subject from the vital information of the subject has been proposed. For example, a biological information processor that detects a plurality of pieces of vital information (breathing, pulse, myoelectricity, and the like) from a subject and estimates the psychological state (arousal level and emotional value) of an audience and the intensity thereof from the detected measurement values and the initial values or standard values thereof is known (see PTL 1).

However, in a case where a plurality of contact-type sensors and non-contact type sensors are required to detect the subject's vital information, the processor becomes complicated and the cost increases. In particular, the use of a contact-type sensor is annoying to the subject. In addition, in a case where there are a plurality of subjects, sensors are required for the number of people, thus the processor becomes more complicated and the cost increase.

If the viewing state (degree of concentration, drowsiness, and the like) of the audience viewing a certain content may be associated with the temporal information of the content, it is possible to evaluate the description of the content, which is useful.

According to the present disclosure, it is possible to detect the viewing state of the audience viewing the content with a simple configuration and to associate the detected viewing state with temporal information of the content.

### CITATION LIST

### Patent Literature

PTL 1: JP-A-2006-6355

### SUMMARY OF THE INVENTION

The viewing state detection device of the present disclosure is a viewing state detection device that detects a viewing state of an audience from images including the audience viewing a content including an image input unit to which temporally consecutive captured images including the audience and information on the captured time of the captured images are input, an area detector that detects a skin area of the audience from the captured images, a vital information extractor that extracts vital information of the audience based on the time-series data of the skin area, a viewing state determination unit that determines the viewing state of the audience based on the extracted vital information, a content information input unit to which content information including at least temporal information of the content is input, and a viewing state storage unit that stores the viewing state in association with the temporal information of the content.

According to the present disclosure, it is possible to detect the viewing state of the audience viewing the content with a simple configuration and to associate the detected viewing state with the temporal information of the content.

### BRIEF DESCRIPTION OF DRAWINGS

FIG. 1 is an overall configuration diagram of a viewing state detection system according to a first embodiment.

FIG. 2 is a functional block diagram of the viewing state detection system according to the first embodiment.

FIG. 3 is an explanatory diagram of pulse wave extraction processing with a viewing state detection device in FIG. 2.

FIG. 4 is an explanatory diagram of pulse wave extraction processing with the viewing state detection device in FIG. 2.

FIG. 5 is a diagram showing an example of vital information.

FIG. 6 is a diagram showing an example of content information.

FIG. 7 is a diagram showing an example in which vital information and content information are associated with each other with an elapsed time of a content.

FIG. 8 is a diagram showing an example of determination information.

FIG. 9A is a diagram showing an example of an output of a viewing state.

FIG. 9B is a diagram showing an example of the output of the viewing state.

FIG. 10 is a flowchart showing a flow of processing by the viewing state detection device according to the first embodiment.

FIG. 11 is an overall configuration diagram of a viewing state detection system according to a second embodiment.

FIG. 12 is a functional block diagram of a viewing state detection device according to a third embodiment.

FIG. 13 is a functional block diagram of a viewing state detection device according to a fourth embodiment.

FIG. 14 is a functional block diagram of a viewing state detection device according to a fifth embodiment.

FIG. 15 is a functional block diagram of a viewing state detection device according to a sixth embodiment.

### DESCRIPTION OF EMBODIMENTS

Hereinafter, embodiments of the present disclosure will be described in detail with reference to drawings as appropriate.

### Embodiment 1

<Structure of Face Identification Device>

Embodiments of the present disclosure will be described with reference to drawings.

### First Embodiment

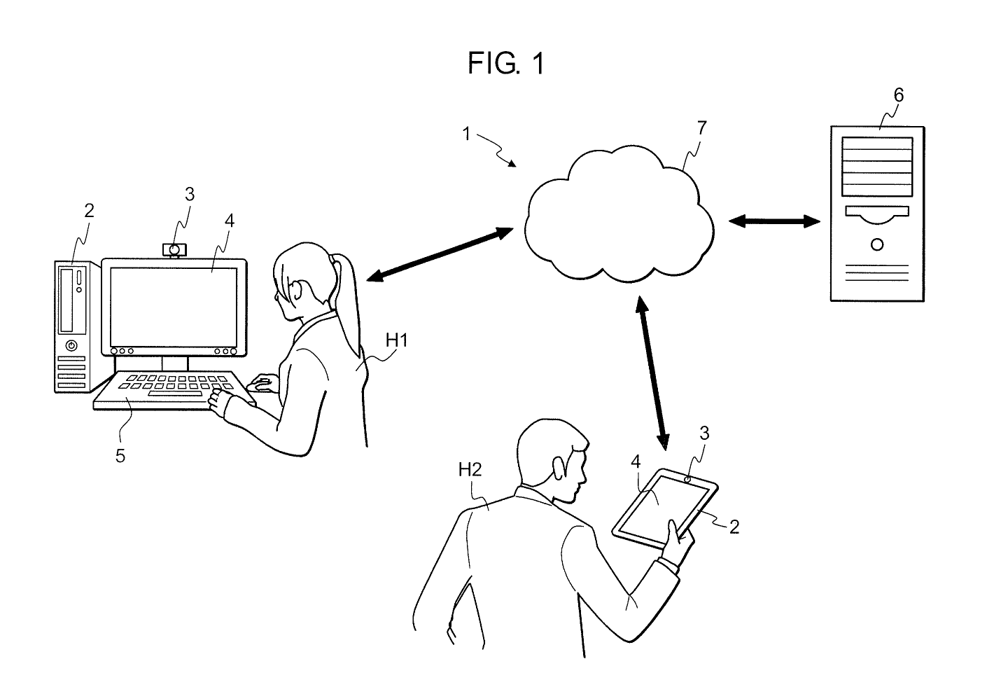

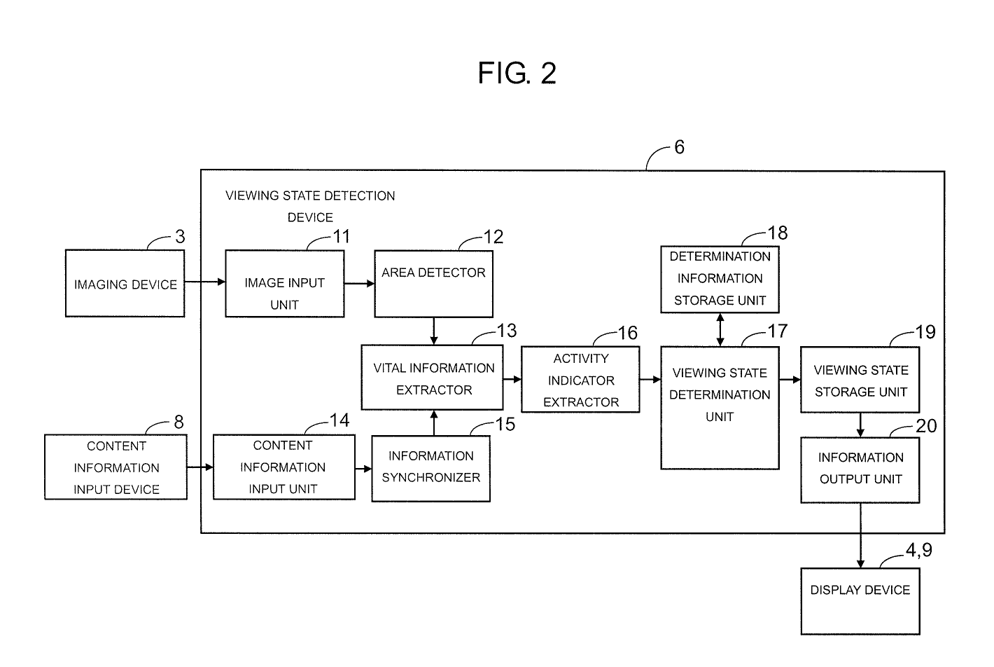

FIGS. 1 and 2 are an overall configuration diagram and a functional block diagram of viewing state detection system 1 according to a first embodiment of the present disclosure, respectively. This first embodiment shows an example in which the viewing state detection system according to the present disclosure is applied to e-learning. That is, the viewing state detection system 1 according to the first embodiment is used for detecting the viewing state (degree of concentration and drowsiness) of an audience of e-learning.

As shown in FIG. 1, the viewing state detection system 1 according to the first embodiment of the present disclosure, personal computer 2 or tablet 2 used by audience H1 and H2 of e-learning (hereinafter, referred to as reference sign H when collectively used), imaging device (camera) 3 that images at least a part of audience H, display screen 4 that displays the content of e-learning or display screen 4 of tablet 2, a keyboard 5 for operating personal computer 2, and viewing state detection device 6. In addition, although not shown in FIG. 1, as shown in FIG. 2, viewing state detection system 1 further includes content information input device 8 and display device 9.

Camera 3 and viewing state detection device 6 are communicably connected via network 7 such as the Internet or a local area network (LAN). Imaging device 3 and viewing state detection device 6 may be directly connected so as to communicate with each other by a known communication cable. Likewise, content information input device 8 and display device 9 are communicably connected to viewing state detection device 6 via network 7 or by a known communication cable.

The camera 3 is a camera having a well-known configuration and forms light from an object (audience H) obtained through a lens on an image sensor (CCD, CMOS, and the like) which is not shown), thereby outputting a video signal obtained by converting the light of the formed image into an electric signal to viewing state detection device 6. For camera 3, a camera attached to personal computer 2 or tablet 2 of audience H may be used, or a separately prepared camera may be used. It is also possible to use an image storage device (image recorder) which is not shown instead of camera 3 and to input the recorded image of audience H during the viewing of the content from the image storage device to viewing state detection device 6.

Content information input device 8 is for inputting content information including at least temporal information of the content to viewing state detection device 6. Specifically, as temporal information of the content, it is preferable to use elapsed time since the start of the content.

As described above, display screen 4 is display device 4 of audience H1 or display screen 4 of tablet 2 of audience H2, and display device 9 is, for example, a display device of a content provider. On display devices 4 and 9, the audience state detected by viewing state detection device 6 is displayed. In the present embodiment, the audience state is a degree of concentration and drowsiness of audience H. It is also possible to use a sound notification device which can notify audience state by voice or sound together with display device 9 or instead of display device 9.

The viewing state detection device 6 may extract vital information (here, a pulse wave) of audience H of the content based on the captured images input from imaging device 3 and associate the extracted vital information and the content information with the captured time of the captured images and the temporal information of the content. Then, viewing state detection device 6 may determine the viewing state (degree of concentration and drowsiness) of audience H based on the extracted vital information and notify audience H and the content provider of the determined viewing state of audience H together with the content information. In addition, when a plurality of audience H exist, viewing state detection device 6 may notify audience H's viewing state as the viewing state of each audience, or the viewing state of all or a part of the people.

As shown in FIG. 2, viewing state detection device 6 includes image input unit 11 to which temporally consecutive captured images including at least a part of audience H currently viewing the content from imaging device 3 and information on the captured time of the captured images are input, area detector 12 that detects a skin area (in this case, a face area) of audience H from the captured images, vital information extractor 13 that extracts the vital information of audience H based on the detected time-series data of the skin area of audience H, content information input unit 14 to which content information including at least temporal information of the content is input from content information input device 8, and information synchronizer 15 that associates the vital information and the content information with the captured time of the captured images and the temporal information of the content.

Further, viewing state detection device 6 includes activity indicator extractor 16 that extracts physiological or neurological activity indicators of audience H from the extracted vital information, viewing state determination unit 17 that determines the viewing state of audience H based on the extracted activity indicators, determination information storage unit 18 that stores the determination information used for the determination, viewing state storage unit 19 that stores the determined viewing state of audience H in association with the content information, and information output unit 20 that outputs the viewing state and content information of audience H stored in viewing state storage unit 19 to display devices 4 and 9. Each unit is controlled by a controller (not shown).

Image input unit 11 is connected to imaging device 3, and temporally consecutive captured images (data of frame images) including at least a part of audience H during the viewing of the content are input from imaging device 3 as video signals. In addition, information on the captured time of the captured images is also input to image input unit 11. The captured time is the elapsed time since imaging of audience H started, and is associated with the captured image. In the present embodiment, it is assumed that imaging of audience H starts at the start of playing of the e-learning content. Therefore, the captured time is the same as the elapsed time from the start of playing of the content. The captured images input to image input unit 11 are sent to area detector 12.

The area detector 12 executes face detection processing based on a well-known statistical learning technique using facial feature quantities with respect to each captured image (frame image) acquired from image input unit 11, thereby detecting and tracking the detected face area as the skin area of audience H and obtaining information on the skin area (the number of pixels constituting the skin area). The information on the skin area acquired by area detector 12 is sent to vital information extractor 13. For the skin area detection processing by area detector 12, in addition to the well-known statistical learning method using facial feature quantities, face detection processing based on a known pattern recognition method (for example, matching with a template prepared in advance) may be used. In addition, in a case a plurality of images of audience H are included in the captured images acquired from image input unit 11, it is assumed that area detector 12 extracts target audience H using a known detection method and perform the above processing on extracted audience H.

Vital information extractor 13 calculates the pulse of audience H based on the skin area of the captured images obtained from area detector 12. More specifically, for example, pixel values (0 to 255 gradations) of each component of RGB are calculated with respect to each pixel constituting the skin area extracted in the temporally consecutive captured images to generate time-series data of the representative value (here, the average value of each pixel) as a pulse signal. In this case, the time-series data may be generated based on the pixel value of only the green component (G) of which variation is particularly large due to the pulsation.

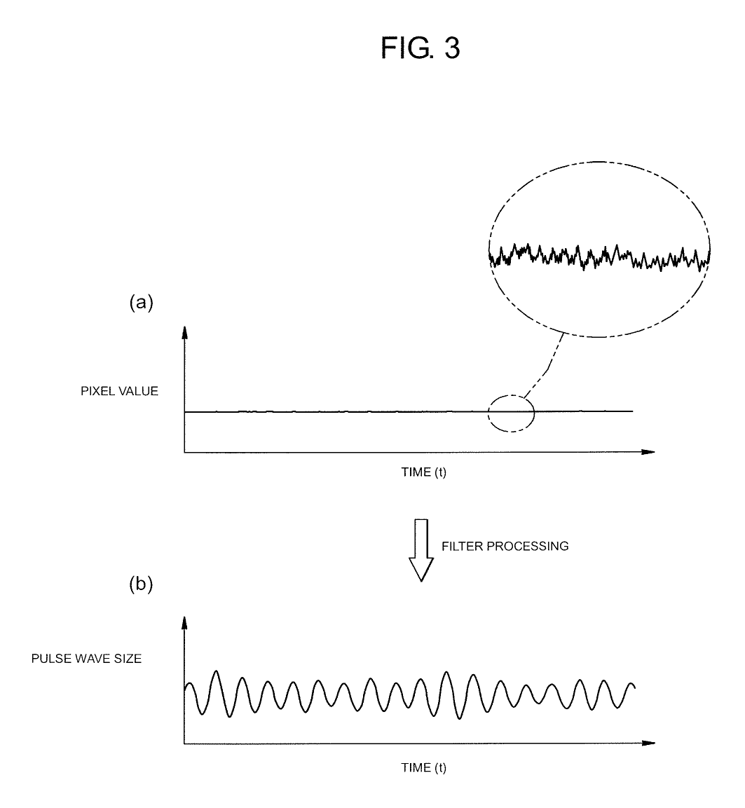

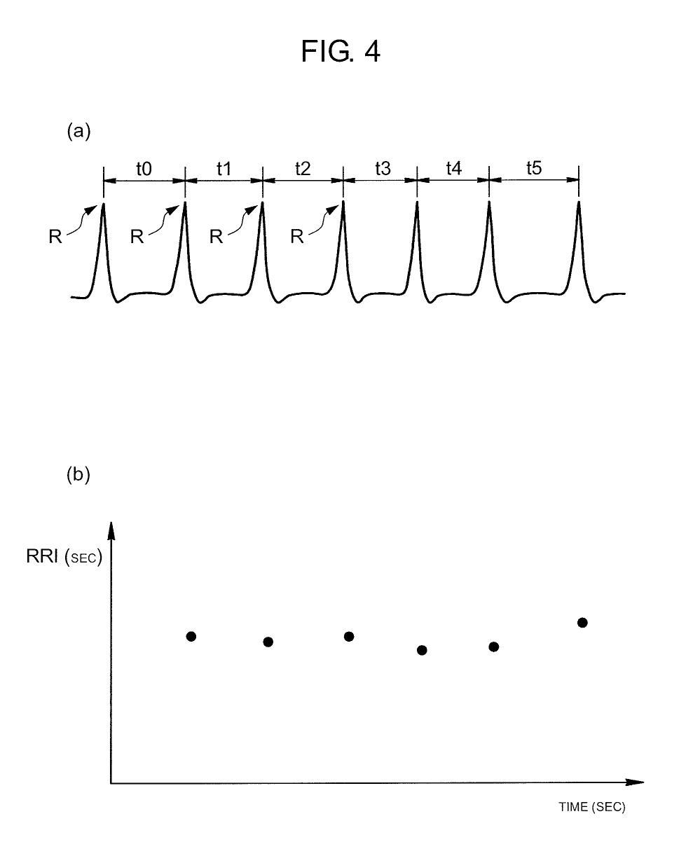

For example, as shown in FIG. 3(a), the time-series data of the generated pixel value (average value) is a minute variation based on a change in the hemoglobin concentration in the blood (for example, a variation of pixel value less than one grayscale). Therefore, vital information extractor 13 extracts the pulse wave from which noise component is removed as a pulse signal by performing known filter processing (for example, processing by a band pass filter in which a predetermined band pass is set) on the time-series data based on the pixel value, as shown in FIG. 3(a). Then, as shown in FIG. 4(a), vital information extractor 13 calculates a pulse wave interval (RRI) from the time between two or more adjacent peaks in the pulse wave and uses the RRI as the vital information. As described above, since the captured time is associated with the captured image, the vital information extracted from the captured image is also associated with the captured time. The vital information (RRI) extracted by vital information extractor 13 is sent to activity indicator extractor 16.

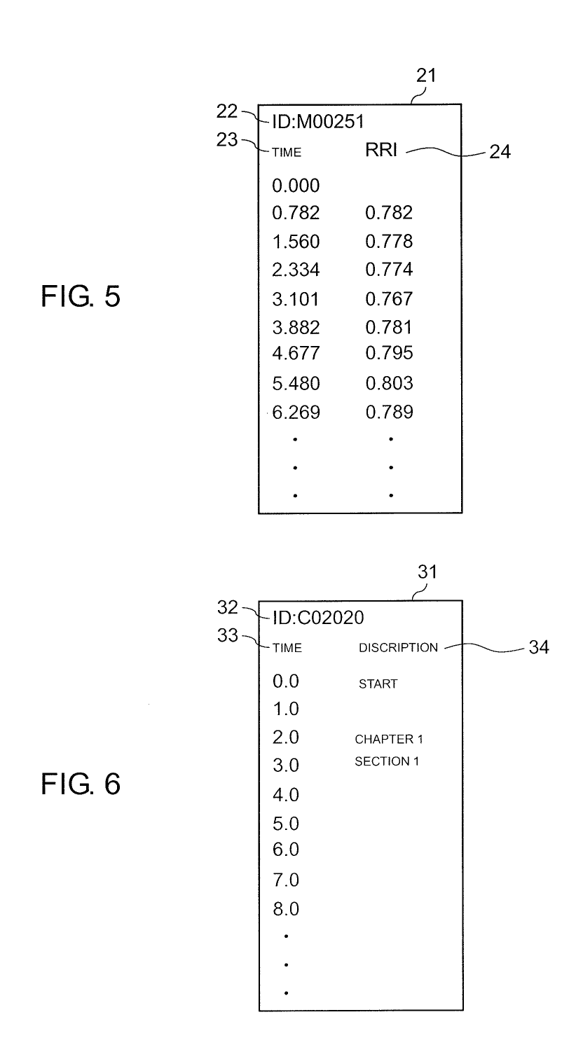

FIG. 5 shows an example of the vital information of audience H1 extracted by vital information extractor 13. As shown in FIG. 5, vital information 21 includes ID number 22 of audience H 1, captured time 23 of the captured images, and RRI value 24 at each captured time 23. ID number 22 (in this example, ID: M00251) of audience H1 is given by vital information extractor 13 to identify audience H. ID number 22 gives a number unrelated to personal information such as the member ID of audience H and the like and audience H may know ID number 22 given to himself/herself, but it is desirable that the content provider may not be able to know the correspondence between audience H and ID number 22. In this way, it is possible to protect audience H's personal information (member ID, vital information, and the like) from the content provider or a third party. As described above, captured time 23 is the elapsed time since imaging of audience H started. In the example of FIG. 5, the captured time 23 is “0.782”, “1.560”, “2.334”, . . . when RRI 24 is “0.782”, “0.778”, “0.774”, . . . .

Content information input unit 14 is connected to content information input device 8, and content information including at least the temporal information of the content is input from content information input device 8.

FIG. 6 shows an example of content information of audience H1 input to content information input unit 14. As shown in FIG. 6, content information 31 includes ID number 32 of the content, elapsed time 33 from the start of playing of the content, and content description 34 at each elapsed time 33. Content ID number 32 (in this example, ID: C02020) is given by content information input unit 14 to identify the content. In the example of FIG. 6, content description 34 when elapsed time 33 is “0.0” is “start”, and content description 34 when the elapsed time 33 is “2.0” is “Chapter 1 section 1”.

Information synchronizer 15 is connected to vital information extractor 13 and content information input unit 14 and associates (links) vital information 21 and content information 31 with captured time 23 and elapsed time 33 of the content. As described above, in the present embodiment, since imaging of audience H starts at the start of playing of the e-learning content, captured time 23 (see FIG. 5) of the captured images and elapsed time 33 (see FIG. 6) of the content are the same. Therefore, it is possible to associate vital information 21 and content information 31 with captured time 23 and elapsed time 33 of the content. Specifically, elapsed time 33 of the content and content description 34 (see FIG. 6) are associated with RRI 24 (see FIG. 5) of vital information 21.

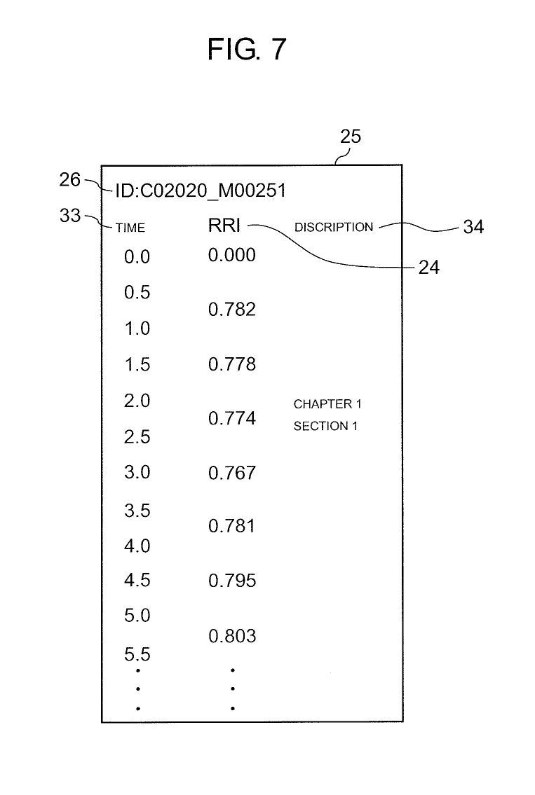

FIG. 7 shows an example in which elapsed time 33 and content description 34 of the content are associated with vital information 21 of audience H1. As shown in FIG. 7, elapsed time 33 and content description 34 of the content are associated with RRI 24 of vital information 21. In this way, it is possible to associate content information 31 with vital information 21. that is, to synchronize vital information 21 with content information 31. As a result, vital information 25 after synchronization with the content information is temporal data including elapsed time 33 of the content. In addition, in the example of FIG. 7, ID number 26 of vital information 25 after synchronization with the content information is ID: C02020_M00251. C02020 is a number for identifying the content, and M00251 is a number for identifying audience H. In the present embodiment, elapsed time 33 of the content is used to synchronize vital information 21 and content information 31, but instead of elapsed time 33 of the content, the time at the time of viewing the content may be used.

Activity indicator extractor 16 extracts the physiological or neurological activity indicators of audience H from the vital information (RRI) acquired from vital information extractor 13. The activity indicators include RRI, SDNN which is a standard deviation of RRI, heart rate, RMSSD or pNN50 which is an indicator of vagal tone intensity, LF/HF which is an indicator of stress, and the like. Based on these activity indicators, it is possible to estimate the degree of concentration and the drowsiness. For example, temporal changes in RRI are found to reflect sympathetic and parasympathetic activity. Therefore, as shown in the graph of FIG. 4(b), it is possible to estimate the degree of concentration, drowsiness, tension (stress), and the like based on the temporal changes of RRI over time, that is, the fluctuation of RRI. The activity indicators extracted by activity indicator extractor 16 are sent to viewing state determination unit 17.

Viewing state determination unit 17 determines the viewing state of audience H based on the activity indicators acquired from activity indicator extractor 16. In the present embodiment, it is assumed that the viewing state is the degree of concentration and the drowsiness. The viewing state is not limited thereto, and various other states such as tension may be used. Specifically, the viewing state of audience H is determined by referring to the determination information indicating a relationship between the temporal changes of the activity indicators and the viewing state (degree of concentration and drowsiness) stored in advance in determination information storage unit 18. As described above with reference to FIG. 7, since vital information 25 after synchronization with the content information is time-series data including elapsed time 33 of the content, the activity indicators extracted from synchronized vital information 25 includes temporal information. Therefore, it is possible to calculate the temporal changes in the activity indicators.

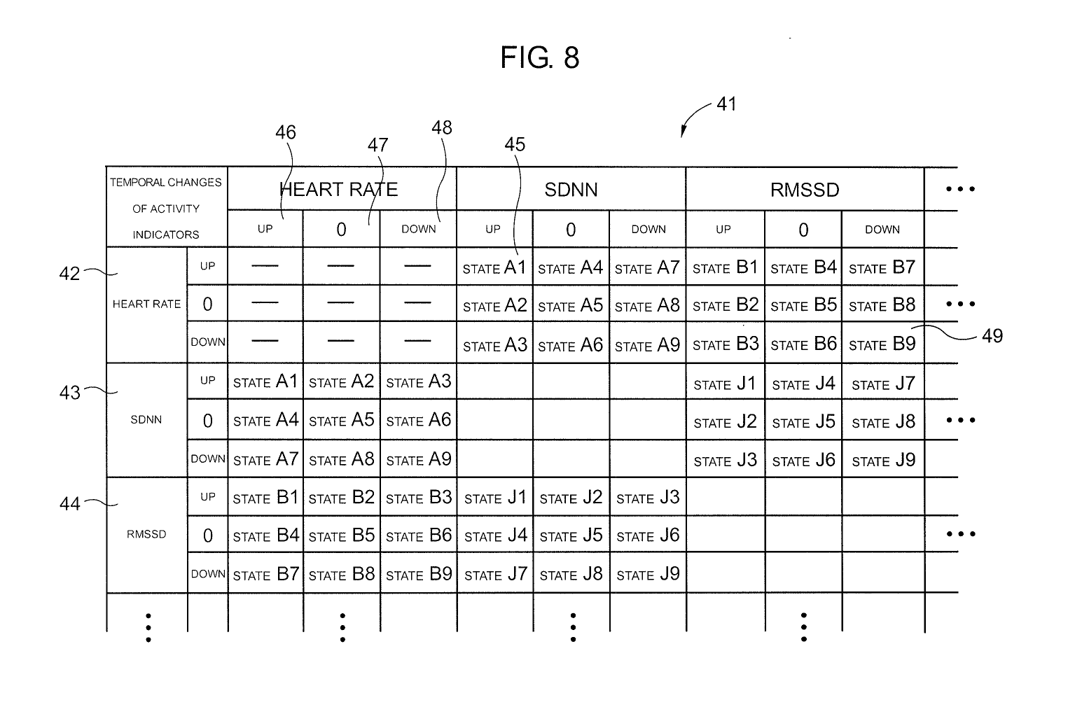

FIG. 8 shows an example of determination information stored in advance in determination information storage unit 18. As shown in FIG. 8, determination information 41 is configured as a table showing the relationship between the temporal changes of heart rate 42, SDNN 43, RMSSD 44, which are the activity indicators, and viewing state 45. The temporal changes in each activity indicator are divided into three stages of “increase (up)” 46, “no change (0)” 47, “decrease (down)” 48, and a combination of two temporal changes of heart rate 42, SDNN 43, and RMSSD 44 is configured to correspond to specific viewing state 45. For example, in a case where heart rate 42 decreases over time and RMSSD 44 decreases over time, viewing state 45 is “state B9” 49. Therefore, if viewing state 45 corresponding to state B9 is known beforehand by a learning method, an experimental method, or the like, viewing state 45 of audience H may be determined based on the temporal changes of heart rate 42 and RMSSD 44. For example, the viewing state of “state B9” is known to be “drowsiness” by a learning or experimental method. Therefore, when heart rate 42 decreases over time and RMSSD 44 decreases over time, it may be determined that the viewing state of audience H is an occurrence of drowsiness. The viewing state determined by viewing state determination unit 17 is sent to viewing state storage unit 19.

Viewing state storage unit 19 stores the viewing state acquired from viewing state determination unit 17 in association with the content information. As described above with reference to FIG. 7, since the vital information is associated with the content information, the viewing state of audience H determined based on the vital information is also associated with the content information. Therefore, the determined viewing state of audience H is stored in viewing state storage unit 19 as temporal data associated with elapsed time 33 of the content (see FIG. 7).

Information output unit 20 is connected to viewing state storage unit 19 and may output the viewing state and content information of audience H stored in viewing state storage unit 19 to display device 4 of audience H or display device 9 of the contents provider. Specifically, information output unit 20 may output the temporal data of the degree of concentration and the drowsiness of audience H to display devices 4 and 9.

In addition, when there are a plurality of audience H information output unit 20 may output the viewing states of the plurality of audience H as the viewing state of each audience or may output the viewing states as a viewing state for all or a part of the plurality of people to display devices 4 and 9. The viewing state for all or a part of the plurality of people may use a ratio or an average value of the degree of viewing state (degree of concentration degree and drowsiness) of each audience.

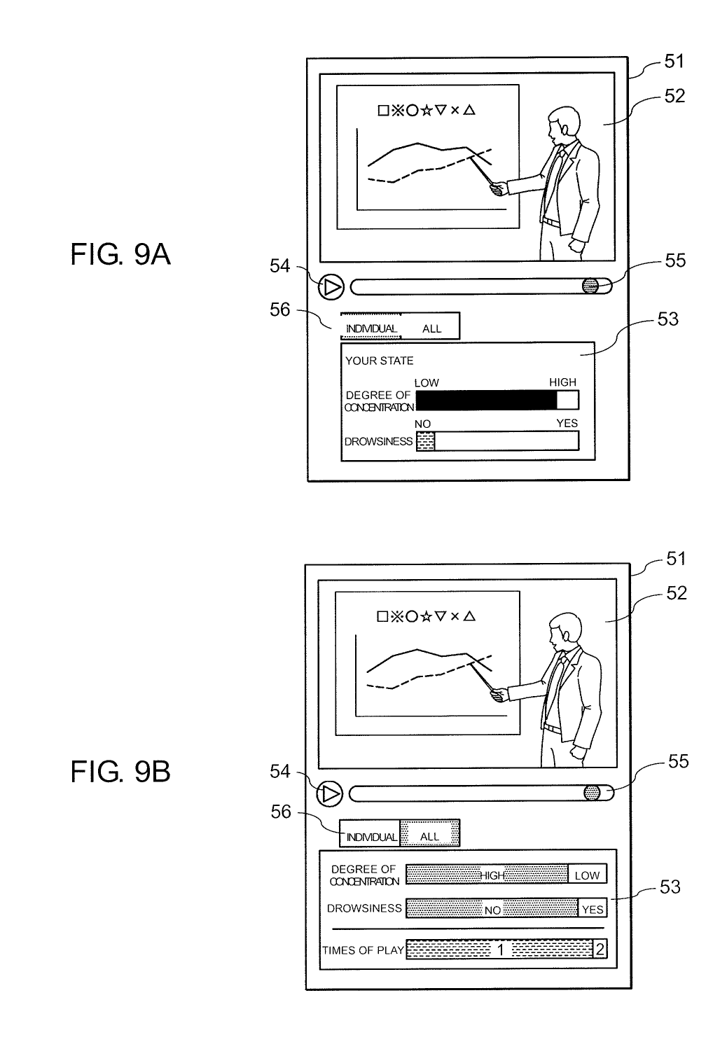

FIG. 9A shows an example in which the temporal data of the degree of concentration and the drowsiness of audience H is output to display device 4 of audience H or display device 9 of the contents provider. As shown in FIG. 9A, content play screen 52 is provided on the upper side of screen 51 of display device 4, and viewing state display screen 53 is provided on the lower side of screen 51. In addition, between content play screen 52 and viewing state display screen 53, content play button 54 and time bar 55 indicating elapsed time after the content is played are provided. In addition, between content play button 54 and viewing state display screen 53, selection button 56 for selecting a display target of the viewing state as either an individual or all is provided. In FIG. 9A, the display target of the viewing state is selected by an individual.

In content play screen 52, an image of the content of e-learning is displayed, and on viewing state display screen 53, the degree of concentration and the drowsiness of audience H viewing the content are displayed. The degree of concentration and the drowsiness are indicated by a ratio. In the example of FIG. 9A, the degree of concentration is about 85% and the drowsiness is about 15%. The display of viewing state display screen 53 is updated at predetermined time intervals. For example, when the content is a still image having a predetermined time length, the display on viewing state display screen 53 may be updated in accordance with the timing of switching the still image. In this way, it is possible to display the viewing state (degree of concentration and drowsiness) of audience H in real time for audience H or the contents provider of e-learning.

FIG. 9B shows an example in which the display target of the viewing state is selected as a whole by operating select button 56, and on viewing state display screen 53, the viewing state of all the audience H (hereinafter, also referred to as “all the audience”) of a plurality of people is displayed. Specifically, the ratio of the number of people with a high degree of concentration and people with a low degree of concentration in all the audience and the ratio of the number of people with drowsiness and people without drowsiness are shown. In the example of FIG. 9B, the ratio of the number of people with a high degree of concentration is about 80%, and the ratio of the number of people with a low degree of concentration is about 20%. In addition, the ratio of the number of people with drowsiness is about 85%, and the ratio of the number of people without drowsiness is about 15%. In addition, viewing state display screen 53 also shows the ratio of the number of times that the content is played in all the audience of the content of e-learning. In the example of FIG. 9B, the ratio of people who played one time is about 90%, and the ratio of people who played two times is about 10%. In this way, it is possible to display the viewing state (degree of concentration and drowsiness) of the audience as a whole in real time for audience H or the contents provider of e-learning. In the example of FIG. 9B, the viewing state for all the audience H of plural people is displayed, but it is possible to display the viewing state for a part of all the audience H of the plurality of people.

In addition, temporal data on the degree of concentration and drowsiness of each audience H or the plurality of audience H may be output to display device 9 of the contents provider at a desired point in time after the end of playing of the content. In this case, it is possible to verify the temporal changes in the degree of concentration or the drowsiness of each audience H or the plurality of audience H at each point in time after the end of playing of the content. In this way, it is possible to estimate the content that audience H showed interest, the length of time that audience H may concentrate, and so on. In addition, based on the estimation result, it is also possible to evaluate the quality and the like of the content description and to improve the content description. In addition, in a case where a test for measuring a degree of comprehension of the content description is performed for each audience H after the playing of the content ends, it is also possible to estimate the degree of comprehension of each audience H by comparing the result of the test with the viewing state (degree of concentration, drowsiness) of each audience H detected by viewing state detection device 6. In this case, audience H may read the viewing state information from viewing state storage unit 19 using the ID number, and audience H may compare the test result and the viewing state by himself or herself. Then, the comparison result (degree of comprehension) may be notified to the contents provider. In this way, it is possible to protect the personal information of audience H (member ID, viewing state information, test results, and the like). According to viewing state detection system 1 according to the first embodiment of the present disclosure, it is not necessary to attach a contact type sensor to audience H, thus audience H does not feel annoyed.

Viewing state detection device 6 as described above may consist of an information processing device such as a personal computer (PC), for example. Although not shown in detail. viewing state detection device 6 includes a hardware configuration including a central processing unit (CPU) that comprehensively executes various kinds of information processing and control of peripheral devices based on a predetermined control program, a random access memory (RAM) that functions as a work area of the CPU, a read only memory (ROM) that store control programs and data executed by the CPU, a network interface for executing communication processing via network, a monitor (image output device), a speaker, an input device, and a hard disk drive (HDD), and at least a part of the functions of each unit of viewing state detection device 6 shown in FIG. 2 may be realized by the CPU executing a predetermined control program. At least a part of the functions of viewing state detection device 6 may be replaced by another known hardware processing.

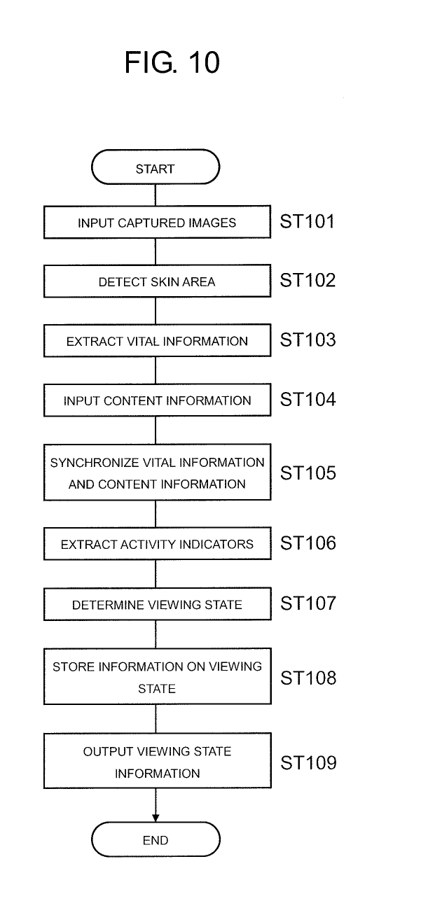

FIG. 10 is a flowchart showing a flow of processing by viewing state detection device 6 according to the first embodiment.

First, temporally consecutive captured images including audience H and information on the captured time of the captured images are input to image input unit 11 (ST 101). Area detector 12 detects the skin area of audience H from the captured images (ST 102), and vital information extractor 13 extracts the vital information of audience H based on the time-series data of the skin area (ST 103).

Next, content information including at least the temporal information of the content is input to content information input unit 14 (ST 104), and information synchronizer 15 associates the content information and the vital information with captured time of the captured images and temporal information of the content (ST 105). In the present embodiment, since imaging of audience H starts from the start of play of the content, captured time is the same as the elapsed time of the content. Therefore, the content information and the vital information may be associated with the temporal information of the content. That is, the content information and the vital information may be synchronized.

Next, activity indicator extractor 16 extracts the physiological or neurological activity indicator of audience H from the vital information extracted by vital information extractor 13 (ST 106). Subsequently, viewing state determination unit 17 refers to the determination information stored in determination information storage unit 18 based on the activity indicator extracted by activity indicator extractor 16 to determine the viewing state of audience H (ST 107). The information of the viewing state determined by viewing state determination unit 17 is stored in viewing state storage unit 19 (ST 108).

Then, the information of the viewing state stored in viewing state storage unit 19 is output from information output unit 20 to display device 4 of audience H or display device 9 of the contents provider (ST 109).

In viewing state detection device 6, the above-described steps ST 101 to ST 109 are repeatedly executed on the captured images sequentially input from imaging device 3.

### Second Embodiment

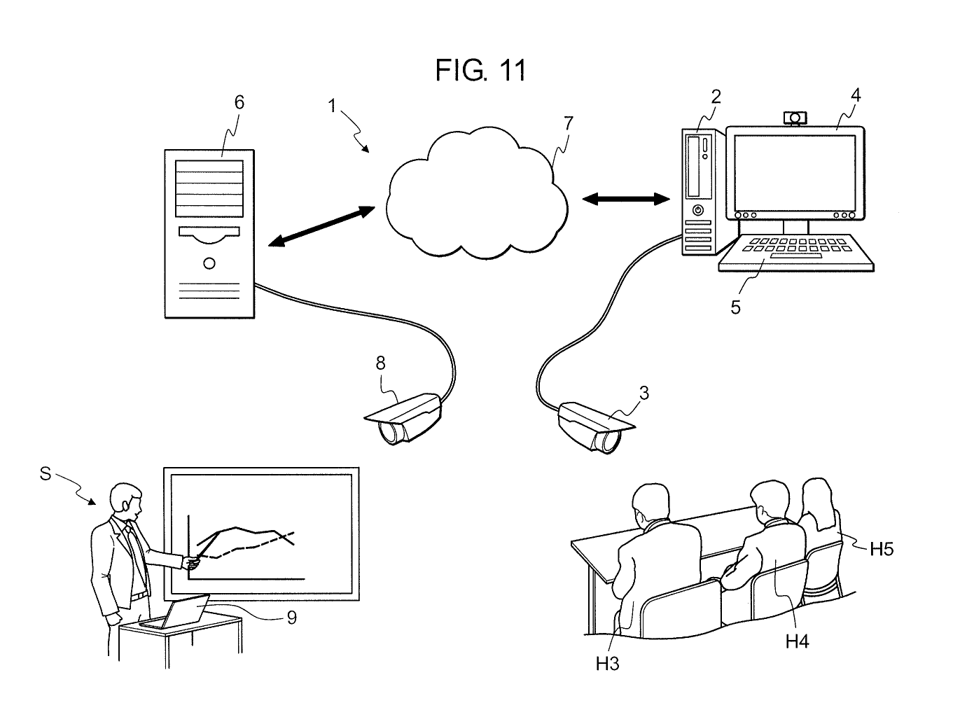

FIG. 11 is an overall configuration diagram of a viewing state detection system according to a second embodiment of the present disclosure. This second embodiment shows an example in which the viewing state detection system according to the present disclosure is applied to a lecture. In FIG. 10, the same reference numerals are given to the same constituent elements as those of the above-described first embodiment. In addition, in the second embodiment, matters not specifically mentioned below are the same as those in the case of the first embodiment described above.

This second embodiment is used for detecting the viewing state of audience H viewing the lecture. In addition, in this second embodiment, a camera is used as content information input device 8. The description (content) of speaker S is captured by camera 8, and the captured images are input to content information input unit 14 (see FIG. 2) of viewing state detection device 6 together with the temporal information of the content.

A plurality of audiences H (H3, H4, and H5) are imaged by camera (imaging device) 3. In a case where audiences H3, H4, and H5 fall within the imaging visual field of camera 3, the audiences may be imaged at the same time. In that case, in area detector 12 of viewing state detection device 6, each audience H is extracted. In addition, audiences H3, H4, and H5 may alternatively be captured by sequentially changing the capturing angle of camera 3 using a driving device (not shown). As a result, it is possible to capture audiences H3, H4, and H5 almost at the same time. The images of each audience H imaged by camera 3 are input to image input unit 11 (see FIG. 2) of viewing state detection device 6 for each audience. Thereafter, for each audience, the same processing as that in the case of the above-described first embodiment is performed. As in the first embodiment, it is assumed that capturing of audience H starts from the start of the lecture (content).

In addition, as display device 9 of the contents provider, a laptop computer is installed in front of speaker S, and viewing state detection device 6 sends temporal data of the degree of concentration and drowsiness on all the audience to notebook personal computer 9. As a result, the display screen as shown in FIG. 9B is displayed on the display screen of notebook personal computer 9. As a result, speaker S may visually recognize the temporal data of the degree of concentration and drowsiness on all the audience in real time and it is possible to deal with the concentration and drowsiness in all the audience on the spot. For example, in a case where the ratio of people with a low degree of concentration is increased in all the audience or in a case where the ratio of people with drowsiness in all the audience increases, it is possible to change the way of speaking (the tone of voice, the size of voice) and the lecture content as appropriate so as to attract the interest of audience H.

In addition, as in the first embodiment, the temporal data on the degree of concentration and drowsiness of each audience H or the plurality of audience H may be output to display device 9 of the contents provider at a desired point in time after the end of playing of the content. As a result, after the lecture is over, it is possible to verify the temporal changes of the degree of concentration and drowsiness of each audience H or the plurality of audience H at each point in the content of the lecture. In this way, it is possible to estimate the content that audience H showed interest, the length of time that audience H may concentrate, and so on. In addition, based on the estimation result, it is also possible to evaluate the quality of lecture content and to improve the lecture content of the next and subsequent lectures. In addition, instead of a lecture, in a case where a lecture or a lesson is provided, in a case where a test for measuring a degree of comprehension of the content description of the lecture or the lesson is performed for each audience H after the lecture or the lesson ends, it is also possible. to estimate the degree of comprehension of each audience H by comparing the result of the test with the viewing state (degree of concentration, drowsiness) of each audience H detected by viewing state detection device 6. In this case, as in the first embodiment, audience H may read information on the viewing state from viewing state storage unit 19 using the ID number, and audience H may compare the test result and the viewing state by himself or herself. Then, the comparison result (degree of comprehension) may be notified to the contents provider. In this way, it is possible to protect the personal information of audience H (member ID, viewing state information, test results, and the like). According to viewing state detection system 1 according to the second embodiment of the present disclosure, it is not necessary to attach a contact type sensor to audience H, thus audience H does not feel annoyed.

### Third Embodiment

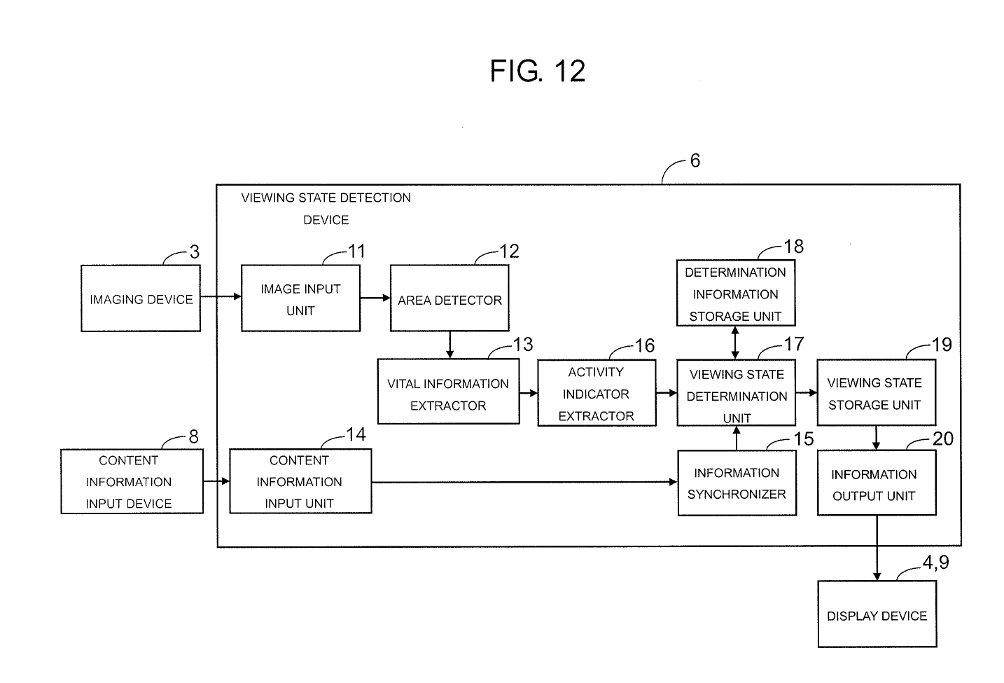

FIG. 12 is a block diagram of viewing state detection device 6 according to a third embodiment of the present disclosure. Viewing state detection device 6 according to the third embodiment differs from viewing state detection device 6 according to the first embodiment shown in FIG. 2 in that information synchronizer 15 is connected not to vital information extractor 13 but to viewing state determination unit 17. Since other configurations are the same as those of the first embodiment, the same components are denoted by the same reference numerals, and description thereof is omitted.

As shown in FIG. 12, information synchronizer 15 is connected to viewing state determination unit 17, the information of the determination result (that is, the viewing state) in viewing state determination unit 17 and content information 31 (see FIG. 6) are associated with the captured time of the captured images and the elapsed time of the content. Since the captured images are associated with the captured time, the viewing state determined based on the activity indicator extracted from the captured images is also associated with the captured time. Then, as described above, in the present embodiment, since capturing of audience H starts at the time of playing or the start of the content, the captured time of the captured images is the same as the elapsed time of the content. Therefore, the determination result (viewing state) in viewing state determination unit 17 and content information 31 may be associated with each other by elapsed time 33 of the content. More specifically, elapsed time 33 of the content and content description 34 (see FIG. 6) are associated with the viewing state of each audience H.

In this way, when information synchronizer 15 is connected to viewing state determination unit 17, the degree of freedom of the configuration of viewing state detection device 6 may be increased, which is useful. For example, when viewing state detection system 1 according to the present disclosure is applied to a lecture (see FIG. 2), it is possible to directly associate the content information (captured images of the lecture) captured by camera (content information input device) 8 with the information of the viewing state determined by viewing state determination unit 17.

### Fourth Embodiment

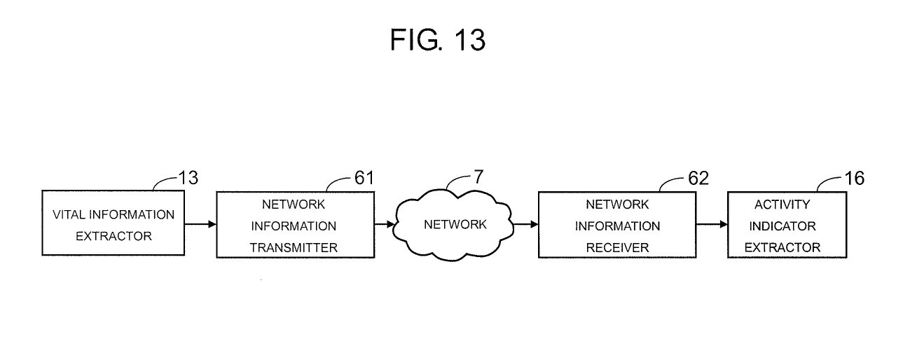

FIG. 13 is a block diagram of viewing state detection device 6 according to a fourth embodiment of the present disclosure. Viewing state detection device 6 according to the fourth embodiment differs from viewing state detection device 6 according to the first embodiment shown in FIG. 2 in that vital information extractor 13 and activity indicator extractor 16 are connected via network 7 such as the Internet, a local area network (LAN), or the like. Since other configurations are the same as those of the first embodiment, the same components are denoted by the same reference numerals, and description thereof is omitted.

As shown in FIG. 13, viewing state detection device 6 further includes network information transmitter 61 and network information receiver 62. Network information transmitter 61 is connected to vital information extractor 13, and network information receiver 62 is connected to activity indicator extractor 16. Network information transmitter 61 transmits vital information 21 (see FIG. 5) extracted by vital information extractor 13 to network information receiver 62 via network 7. Network information receiver 62 receives vital information 21 from network information transmitter 61 via network 7. Vital information 21 received by network information receiver 62 is sent to activity indicator extractor 16.

In this way, when vital information extractor 13 and activity indicator extractor 16 are connected via network 7, the degree of freedom of the configuration of viewing state detection device 6 may be increased, which is useful. For example, when the data of the captured images of audience H captured by camera 3 is transmitted to viewing state detection device 6 via network 7, the amount of data transmitted via network 7 is large, which is undesirable. Therefore, in a case where viewing state detection system 1 according to the present disclosure is applied to e-learning (see FIG. 1), after processing for extracting vital information from the captured images on the personal computer or tablet 2 of audience H is performed, the extracted vital information may be configured to be transmitted to activity indicator extractor 16 via network 7. In this way, when the data of the vital information, not the data of the captured images of audience H, is configured to be transmitted, via network 7, the amount of data transmitted via network 7 may be reduced. Therefore, it is useful when viewing state detection system 1 according to the present disclosure is applied to e-learning. It is equally useful in applying viewing state detection system 1 according to the present disclosure to a lecture.

### Fifth Embodiment

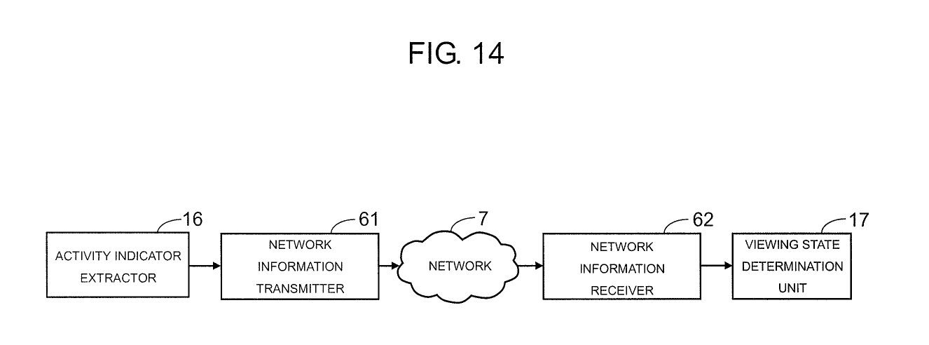

FIG. 14 is a block diagram of viewing state detection device 6 according to a fifth embodiment of the present disclosure. Viewing state detection device 6 according to the fifth embodiment differs from viewing state detection device 6 according to the first embodiment shown in FIG. 2 in that activity indicator extractor 16 and viewing state determination unit 17 are connected via network 7 such as the Internet or a local area network (LAN). Since other configurations are the same as those of the first embodiment, the same components are denoted by the same reference numerals, and description thereof is omitted.

As shown in FIG. 14, viewing state detection device 6 further includes network information transmitter 61 and network information receiver 62. Network information transmitter 61 is connected to activity indicator extractor 16, and network information receiver 62 is connected to viewing state determination unit 17. Network information transmitter 61 transmits the activity indicator extracted by activity indicator extractor 16 to network information receiver 62 via network 7. Network information receiver 62 receives the activity indicator from network information transmitter 61 via network 7. The activity indicator received by network information receiver 62 is sent to viewing state determination unit 17.

In this way, when activity indicator extractor 16 and viewing state determination unit 17 are connected via network 7, the degree of freedom of the configuration of the viewing state detection device 6 may be increased, which is useful. In addition, in this way, by configuring the data of the activity indicator, not the data of the captured images of audience H, to be transmitted via network 7, the amount of data to be transmitted via the network 7 may be reduced. Therefore, as in the case of the above-described fourth embodiment, it is useful when viewing state detection system 1 according to the present disclosure is applied to e-learning. It is equally useful in applying viewing state detection system 1 according to the present disclosure to a lecture.

### Sixth Embodiment

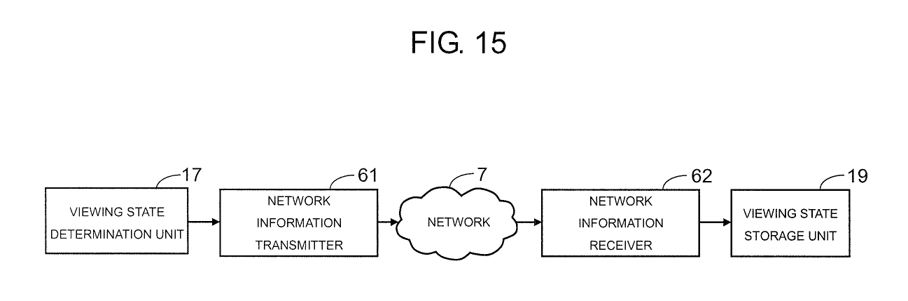

FIG. 15 is a block diagram of viewing state detection device 6 according to a sixth embodiment of the present disclosure. Viewing state detection device 6 according to the sixth embodiment differs from viewing state detection device 6 according to the first embodiment shown in FIG. 2 in that viewing state determination unit 17 and viewing state storage unit 19 are connected via network 7 such as the Internet or a local area network (LAN). Since other configurations are the same as those of the first embodiment, the same components are denoted by the same reference numerals, and description thereof is omitted.

As shown in FIG. 15, viewing state detection device 6 further includes network information transmitter 61 and network information receiver 62. Network information transmitter 61 is connected to viewing state determination unit 17, and network information receiver 62 is connected to viewing state storage unit 19. Network information transmitter 61 transmits information on the viewing state determined by viewing state determination unit 17 to network information receiver 62 via network 7. Network information receiver 62 receives information on the viewing state from network information transmitter 61 via network 7. Information on the viewing state received by network information receiver 62 is sent to viewing state storage unit 19.

In this way, when viewing state determination unit 17 and viewing state storage unit 19 are connected via network 7, the degree of freedom of the configuration of viewing state detection device 6 may be increased, which is useful. In addition, in this way, by configuring the information on the viewing state, not the data of the captured images of audience H, to be transmitted via network 7, the amount of data to be transmitted via the network 7 may be reduced. Therefore, as in the case of the above-described fourth embodiment and the fifth embodiment, it is useful when viewing state detection system 1 according to the present disclosure is applied to e-learning. It is equally useful in applying viewing state detection system 1 according to the present disclosure to a lecture.

The present disclosure relates to a viewing state detection device that detects a viewing state of an audience from images including the audience viewing a content and includes an image input unit to which temporally consecutive captured images including the audience and information on the captured time of the captured images are input, an area detector that detects a skin area of the audience from the captured images, a vital information extractor that extracts vital information of the audience based on the time-series data of the skin area, a viewing state determination unit that determines the viewing state of the audience based on the extracted vital information, a content information input unit to which content information including at least the temporal information of the content is input, and a viewing state storage unit that stores the viewing state in association with the temporal information of the content.

According to this configuration, since the viewing state of the audience is detected based on the audience vital information detected from the images including the audience viewing the content, it is possible to detect the viewing state of the audience viewing the content with a simple configuration. In addition, since the detected viewing state is related to the temporal information of the content, it is possible to evaluate the content description based on the viewing state.

In addition, in the present disclosure, the viewing state may include at least one of the degree of concentration and the drowsiness of the audience.

According to this configuration, since at least one of the degree of concentration of audience and drowsiness is detected, it is possible to estimate the interest and comprehension of the audience for the content based on the degree of concentration and drowsiness of the audience viewing the content.

In addition, the present disclosure may further include an information output unit that outputs viewing state information stored in the viewing state storage unit to an external display device.

According to this configuration, since information on the viewing state stored in the viewing state storage unit is output to the external display device, it is possible to display the viewing state of the audience for the audience or the contents provider. In this way, it is possible for the audience or the contents provider to grasp the viewing state of the audience, and it is also possible to evaluate the content description based on the viewing state of the audience.

In addition, in the present disclosure, the information output unit may output viewing state information as a viewing state of each audience in a case where there are a plurality of audiences.

According to this configuration, the information output unit, in a case where there are the plurality of audiences, since the information of viewing state is configured as information of the viewing state of each audience, it is possible to display the viewing state of each audience for each audience or the contents provider. As a result, each audience or contents provider may grasp the viewing state of each audience in detail.

In addition, the information output unit of the present disclosure may output viewing state information as viewing state information on all or a part of the plurality of people in a case where a plurality of audiences exist.

According to this configuration, in a case a plurality of audiences exist, the information output unit is configured to output viewing state information as information on viewing state of all or a part of the plurality of people, it is possible to display the viewing state on the plurality of people as a whole or the viewing state on a part of the plurality people as a whole for each audience or contents provider. As a result, each audience or contents provider may grasp the viewing state of the plurality of audiences in detail.

In addition, the present disclosure may be a viewing state detection system including a viewing state detection device, an imaging device that inputs captured images to the viewing state detection device, and a content information input device that inputs content information including at least the temporal information of the content.

According to this configuration, it is possible to detect the viewing state of the audience viewing the content with a simple configuration and to associate the detected viewing state with temporal information of the content.

In addition, the present disclosure may further include a display device that displays information on the viewing state output from the viewing state detection device.

According to this configuration, since information on the viewing state output from the viewing state detection device is displayed on the display device, it is possible to display the viewing state of the audience for the audience or the contents provider. In this way, it is possible for the audience or the contents provider to grasp the viewing state of the audience, and it is also possible to evaluate the content description based on the viewing state of the audience.

In addition, the present disclosure relates to a viewing state detection method for detecting a viewing state of an audience from images including the audience viewing a content and may include an image input step of temporally consecutive captured images including the audience and information on the captured time of the captured images being input, an area detection step of detecting a skin area of the audience from the captured images, a vital information extraction step of extracting vital information of the audience based on the time-series data of the skin area, a viewing state determination step of determining the viewing state of the audience based on the extracted vital information, a content information input step of content information including at least the temporal information of the content being input, and a viewing state storage step of storing the viewing state information in association with the temporal information of the content.

According to this method, it is possible to detect the viewing state of the audience viewing the content with a simple configuration and to associate the detected viewing state with temporal information of the content.

Although the present disclosure has been described based on specific embodiments, these embodiments are merely examples, and the present disclosure is not limited by these embodiments. All the constituent elements of the viewing state detection device, the viewing state detection system, and the viewing state detection method according to the present disclosure described in the above embodiment are not necessarily essential. and at least it is possible to select as appropriate without departing from the scope of the present disclosure.

### INDUSTRIAL APPLICABILITY

The viewing state detection device, the viewing state detection system, and the viewing state detection method according to the present disclosure make it possible to detect the viewing state of the audience viewing the content with a simple configuration, and are useful as a viewing state detection device, a viewing-state detection system, a viewing state detection method, and the like that make it possible to associate the detected viewing state with the temporal information of the content.

### REFERENCE MARKS IN THE DRAWINGS

1 VIEWING STATE DETECTION SYSTEM

2 PC, TABLET

3 IMAGING DEVICE (CAMERA)

4 DISPLAY

5 INPUT DEVICE

6 VIEWING STATE DETECTION DEVICE

7 NETWORK

8 CONTENT INFORMATION INPUT DEVICE

9 DISPLAY

11 IMAGE INPUT UNIT

12 AREA DETECTION DEVICE

13 VITAL INFORMATION EXTRACTOR

14 CONTENT INFORMATION INPUT UNIT

15 INFORMATION SYNCHRONIZER

16 ACTIVITY INDICATOR EXTRACTOR

17 VIEWING STATE DETERMINATION UNIT

18 DETERMINATION INFORMATION STORAGE UNIT

19 VIEWING STATE STORAGE UNIT

20 INFORMATION OUTPUT UNIT

H AUDIENCE

S SPEAKER

1. A viewing state detection device that detects a viewing state of an audience from images including the audience viewing a content, the device comprising:
an image input unit to which temporally consecutive captured images including the audience and information on the captured time of the captured images are input;
an area detector that detects a skin area of the audience from the captured images;
a vital information extractor that extracts vital information of the audience based on time-series data of the skin area;
a viewing state determination unit that determines the viewing state of the audience based on the extracted vital information;
a content information input unit to which content information including at least temporal information of the content is input; and
a viewing state storage unit that stores the viewing state in association with the temporal information of the content.

2. The viewing state detection device of claim 1,
wherein the viewing state includes at least one of the degree of concentration and drowsiness of the audience.

3. The viewing state detection device according to claim 1, further comprising:
an information output unit that outputs information on the viewing state stored in the viewing state storage unit to an external display device.

4. The viewing state detection device of claim 3,
wherein the information output unit outputs information on the viewing state as a viewing state of each audience when there are a plurality of audiences.

5. The viewing state detection device of claim 3,
wherein the information output unit outputs information on the viewing state as information on viewing state of all or a part of the plurality of people in a case where there are the plurality of audiences.

6. A viewing state detection system comprising:
the viewing state detection device according to claim 1;
an imaging device that inputs captured images to the viewing state detection device; and
a content information input device that inputs content information including at least temporal information of a content to the viewing state detection device.

7. The viewing state detection system of claim 6, further comprising:
a display device that displays information on viewing state output from the viewing state detection device.

8. A viewing state detection method for detecting a viewing state of an audience from images including the audience viewing a content, the method comprising:
an image input step of inputting temporally consecutive captured images including the audience and information on the captured time of the captured images;
an area detection step of detecting a skin area of the audience from the captured images;
a vital information extraction step of extracting vital information of the audience based on time-series data of the skin area;
a viewing state determination step of determining the viewing state of the audience based on the extracted vital information;
a content information input step of inputting content information including at least temporal information of the content; and
a viewing state storage step of storing information on the viewing state in association with temporal information of the content.
